(19) United States
(12) Patent Application Publication
(10) Pub. No.: US 2023/0077199 A1
(43) Pub. Date: Mar. 9, 2023

(54) PALMITOYL TETRAPEPTIDE-20 CONJUGATED NANOSILVER PARTICLE AND USE THEREOF

(71) Applicant: Chih-Tseng WU, Taichung City (TW)

(72) Inventors: Chih-Tseng WU, Taichung City (TW); Chiu-Yung HONG, Taipei City (TW); Chin-Feng CHAN, Taichung City (TW); Jie-Chen HSU, Taichung City (TW); Ting-Wei CHANG, Taichung City (TW)

(21) Appl. No.: 17/891,646

(22) Filed: Aug. 19, 2022

(30) Foreign Application Priority Data
Aug. 23, 2021 (TW) 110131131

(51) Int. Cl. A61K 8/02; A61K 8/19; A61K 8/64; A61Q 5/10

(52) U.S. Cl. CPC A61K 8/0245; A61K 8/19; A61K 8/64; A61Q 5/10; B82Y 5/00

(57) ABSTRACT

The present invention provides a nanocomposite material comprising a palmitoyl tetrapeptide-20 conjugated nanosilver particle. The nanocomposite material of the present invention has good stability and does not easily deteriorate, and it also exhibits the effects of excellent cutaneous penetration ability, reduced free radical damages, and promotion in melanin synthesis in hair, thereby restoring original hair color and thus solving the problems of premature or mid-aged hair graying.

### CROSS-REFERENCE TO RELATED APPLICATIONS

This application claims priority to Taiwan Patent Application No. 110131131, filed on Aug. 23, 2021. The entire content of the application is hereby incorporated by reference.

### FIELD OF INVENTION

The present invention relates to a nanocomposite material and the use thereof. More specifically, the present invention relates to a nanocomposite material comprising a palmitoyl tetrapeptide-20 conjugated nanosilver particle, which exhibits excellent free radical scavenging ability and cutaneous penetration ability, as well as the use thereof in promoting the synthesis of natural hair pigment.

### BACKGROUND

According to analysis of Statista, the global hair care market size reached USD 92.52 billion in 2020 and is growing gradually, which is expected to reach USD 105.14 billion by 2025. It shows that there is a great need for hair care, such as problems regarding excessive sebum secretion, dandruff, dry and broken hair, hair graying and susceptible to hair loss, etc. Hair or scalp damages may be caused by numerous factors. For example, external factors such as ultraviolet lights, air pollution, excessive combing, hair dyeing or perming, diet, or external injuries and so on, or internal factors such as genetics, etc.

Hair follicle is the basic unit for producing hairs. The hair bulge is located in the upper half part of the hair follicle and in the outer root sheath, and in which contains many stem cells including melanocyte stem cells. Hair bulb, lying at the bottom of the hair root, is a wide spherical structure. The bottom of the hair bulb concaves inward, forming a pear-shape tissue called dermal papilla. Dermal papilla is made of connective tissue and contains blood capillaries, responsible for uptake required nutrients and oxygen. A group of stem cells exists around the dermal papilla called hair matrix, which is formed by epithelial cells and melanocytes. Melanocytes can produce melanin and then the melanin is transferred to the surrounding keratinocytes.

Generally, hair graying may be caused by the following factors: (1) oxidative stress (such as ultraviolet lights, chemicals, etc.) leads to depletion or dysfunction of melanocytes that surrounds the dermal papilla, resulting in reduced melanin production or failing to transfer melanosomes, etc.; (2) genotoxic stress (such as ultraviolet lights, IR, etc.) causes defective melanocyte stem cells in the hair bulge; (3) oxidative or genotoxic stress associated with active hair growth. Research has shown that the diameter of white hair is thicker than black hair as well as the growth rate of white hair is 3 times faster than black hair; (4) lack of growth factors for promoting the formation of black hair, such as α-melanocyte stimulating hormone (α-MSH), adrenocorticotropic hormone (ACTH), β-endorphin, stem cell factor (SCF), hepatocyte growth factor (HGF), and nerve growth factor (NGF), etc. may cause hair graying.

Hair graying is one of the noticeable signs of aging, and many people are eager to recover their original hair color. However, the solution for covering gray hair provided on the current market is merely dyeing the hair artificially, which has many disadvantages such as poor sustainability, long operating time, repeated re-dyeing, unclear color, and scalp irritation caused by chemicals, etc.

In view of these technical problems, the objective of the present invention is to provide a nanocomposite material that is safe and can promote the production of natural hair pigment, thereby restoring the original hair color.

### SUMMARY

### Technical Problems to be Solved

The objective of the present invention is to provide a nanocomposite material, which is formed by conjugation of palmitoyl tetrapeptide-20 with nanosilver particle, thereby improving the problems of palmitoyl tetrapeptide-20 being difficult to be absorb by the skin. The nanocomposite material of the present invention is expected to achieve good cutaneous penetration ability, stimulate production of natural hair pigment, and reduce free radical damages. The present invention provides a viable solution for premature or mid-aged hair graying, and thus restores original hair color.

### Technical Means

In order to achieve the abovementioned objective, the present invention provides the following technical means.

In an aspect, the present invention provides a nanocomposite material, comprising a palmitoyl tetrapeptide-20 conjugated nanosilver particle formed by conjugation of palmitoyl tetrapeptide-20 and nanosilver particle.

In some embodiments, the palmitoyl tetrapeptide-20 conjugated nanosilver particle is analyzed in the form of nanosilver aqueous solution by an ultraviolet/visible spectrophotometer; the absorption spectrum may have a specific absorption spectrum peak within 390 nm to 430 nm, preferably 395 nm to 425 nm, more preferably 400 nm to 420 nm, even more preferably within 405 nm to 415 nm.

In some embodiments, the particle size of the palmitoyl tetrapeptide-20 conjugated nanosilver particle may be within 10 nm to 200 nm, preferably within 10 nm to 150 nm, more preferably within 10 nm to 100 nm, more preferably within 10 nm to 90 nm, even more preferably within 10 nm to 80 nm. In certain embodiments, the mean particle size of the palmitoyl tetrapeptide-20 conjugated nanosilver particle may be within 80 nm to 110 nm, preferably within 85 nm to 105 nm, more preferably within 90 nm to 100 nm.

In some embodiments, the Zeta potential of the palmitoyl tetrapeptide-20 conjugated nanosilver particle may be greater than +15 mV, preferably greater than +20 mV, more preferably greater than +30 mV, even more preferably between +30 mV and +38 mV.

In some embodiments, the palmitoyl tetrapeptide-20 conjugated nanosilver particle has the effects of improving cutaneous penetration ability, reducing free radical damages, and promoting the production of natural hair pigments. In some embodiments, the palmitoyl tetrapeptide-20 conjugated nanosilver particle significantly increases the melanin content in newly born hair. In some embodiments, the palmitoyl tetrapeptide-20 conjugated nanosilver particle increases the melanin synthesis in melanocytes in hair follicle. In some embodiments, the palmitoyl tetrapeptide-20 conjugated nanosilver particle promotes the activity of tyrosinase. In some embodiments, the palmitoyl tetrapeptide-20 conjugated nanosilver particle promotes the expression of melanogenic proteins. In some embodiments, the melanogenic protein is selected from TRP-1, TRP-2, or the combination thereof.

In some embodiments, compared to palmitoyl tetrapeptide-20, the palmitoyl tetrapeptide-20 conjugated nanosilver particle enhances percutaneous absorption efficiency preferably by more than 20 times, more preferably by 20 times to 80 times.

In another aspect, the present invention provides a hair care composition, comprising any nanocomposite material described herein, and the hair care composition ameliorates the gray hair in a subject in need through allowing the newly born hair to recover to its original color.

In another aspect, the present invention provides a method of ameliorating gray hair in a subject in need, comprising the step of contacting the scalp of the subject with any nanocomposite material described herein, thereby increasing the melanin content in newly born hair.

In another aspect, the present invention provides a use of the nanocomposite material as described herein for maintaining natural hair color or ameliorating gray hair appearance in a subject in need.

### Technical Effects

The nanocomposite material comprising palmitoyl tetrapeptide-20 conjugated nanosilver particle described herein has good stability, which can be stored for long term and does not easily deteriorate, and has no cytotoxicity. Applying the nanocomposite material described herein exhibits the effects of excellent cutaneous penetration ability, reduced free radical damages, and promoting melanin synthesis in hair. Established by human subject research, the nanocomposite material described herein prevents the growth of graying hair and restores the subject's hair to their original hair color, and thus provides safe and effective hair care products. Further, the palmitoyl tetrapeptide-20 conjugated nanosilver particle of the present invention improves the poor efficiency of skin absorption of palmitoyl tetrapeptide-20, which increases the percutaneous absorption efficiency to more than 20 times. The penetration of the nanocomposite material described herein can reach to the hair bulb, located at the bottom of hair follicle, and thus can more effectively stimulates the melanocytes to produce melanin, thereby improving the pigment concentration of newly hair born from the hair follicle.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 shows the appearance of a sample of the palmitoyl tetrapeptide-20 conjugated nanosilver particle of the present invention.

FIG. 2 is a distribution graph of the particle size of the palmitoyl tetrapeptide-20 conjugated nanosilver particle of the present invention.

FIG. 3 is a diagram of Zeta potential of the palmitoyl tetrapeptide-20 conjugated nanosilver particle of the present invention.

FIG. 4 is an image of the palmitoyl tetrapeptide-20 conjugated nanosilver particle of the present invention using a transmission electron microscope (TEM); lower left, scale bar=50 nm.

FIG. 5 shows the ultraviolet-visible spectrum of the palmitoyl tetrapeptide-20 conjugated nanosilver particle of the present invention.

FIG. 6 shows the ultraviolet-visible spectrum of the palmitoyl tetrapeptide-20 conjugated nanosilver particle of the present invention after storage for 6 months and 8 months.

FIGS. 7A and 7B depict the appearance of the palmitoyl tetrapeptide-20 conjugated nanosilver particle of the present invention after storage for 6 months (FIG. 7A) and 8 months (FIG. 7B), analyzed by a TEM.

FIGS. 8A and 8B show cell viability of human hair follicle dermal papilla cells (HFDPC) (FIG. 8A) and B16F10 murine melanoma cells (B16F10 cells) (FIG. 8B) after treatment with various concentrations (0.03%, 0.1%, 0.3%, 1% and 3%) of the palmitoyl tetrapeptide-20 conjugated nanosilver particle. Each value is presented as mean±SD, n=3. SD: standard deviation.

FIG. 9 shows melanin content of B16F10 cells after treatment with palmitoyl tetrapeptide-20 conjugated nanosilver particle of the present invention. The melanin content in the treated cells is expressed as percentages compared to the untreated control cells, which is set as 100%. Each value is presented as mean±SD, n=3.

FIG. 10 shows cellular tyrosinase activity after treatment with palmitoyl tetrapeptide-20 conjugated nanosilver particle of the present invention. Cellular tyrosinase activity of the treated cells is expressed as percentages compared to that of the untreated control cells, which is set as 100%. Each value is presented as mean±SD, n=3.

FIG. 11 shows expression levels of melanogenic protein after treatment with palmitoyl tetrapeptide-20 conjugated nanosilver particle of the present invention.

FIG. 12 is a calibration curve showing the relation between hair color levels and lightness values (L value) by linear regression analysis.

FIG. 13 is a comparison picture of the hair color on the left side of a subject's head after using hair care composition of the present invention.

FIG. 14 is a comparison picture of the hair color on the right side of a subject's head after using hair care composition of the present invention.

FIG. 15 is a comparison picture of the hair color on top of a subject's head after using hair care composition of the present invention.

FIG. 16 is a comparison picture of the hair color on a subject's hairline after using hair care composition of the present invention.

### DETAILED DESCRIPTION

The present invention will be further exemplified by the following examples, which are not to be seen as limiting. The embodiments and description are used for illustrating the details and effect of the present invention.

Method of preparing palmitoyl tetrapeptide-20 conjugated nanosilver particle

The tetrapeptide-20 of the palmitoyl tetrapeptide-20 used herein is a peptide sequence derived from α-melanocyte stimulating hormone (α-MSH), enabling the selected sequence to interact with melanocortin 1 receptor (MC1R). The tetrapeptide-20 is a synthetic peptide consisting of arginine, histidine, phenylalanine, and tryptophan.

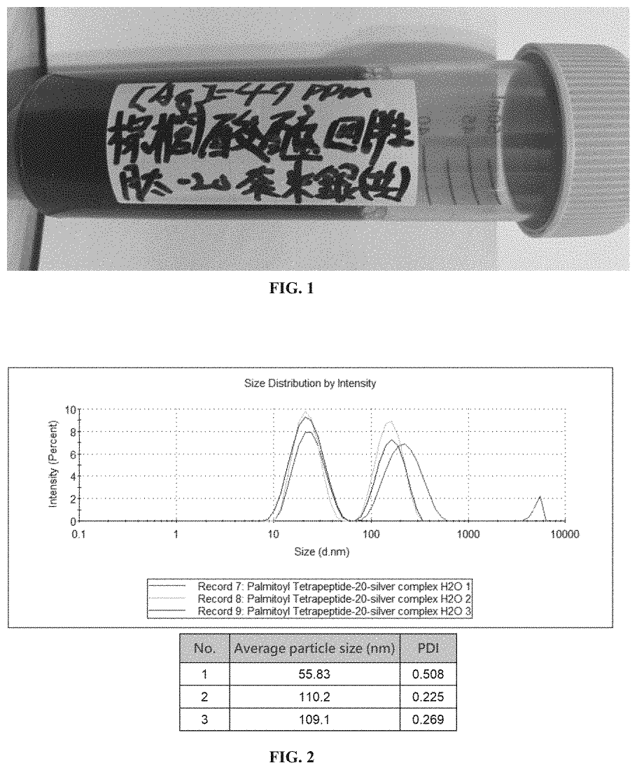

In a 100 mL beaker, 20 mL of AgNO₃ solution (2 mM in water) is mixed with 2 mL of sodium citrate solution (2.5 mM in water) and 23.2 mL of palmitoyl tetrapeptide-20 solution (in concentration of 300 ppm). Subsequently, 1.2 mL freshly prepared NaBH₄ solution (10 mM in ice-cold water) is added to the AgNO₃ solution. The color turns yellow upon addition of NaBH₄, indicating the formation of palmitoyl tetrapeptide-20 conjugated nanosilver particle. The appearance of the sample is shown in FIG. 1.

Analysis of Particles Size Distribution and Zeta Potential

Take 0.1 μL of palmitoyl tetrapeptide-20 conjugated nanosilver particle, add 1980 μL deionized water filtered once with 0.22 μm pore-sized filter, and use Vortex to evenly disperse the palmitoyl tetrapeptide-20 conjugated nanosilver particle. After putting the analysis solution into the sample bottle, use Malvern ZEN 3600 Zetasizer to analyze the particle size distribution and Zeta potential. The analysis is repeated for three independent experiments.

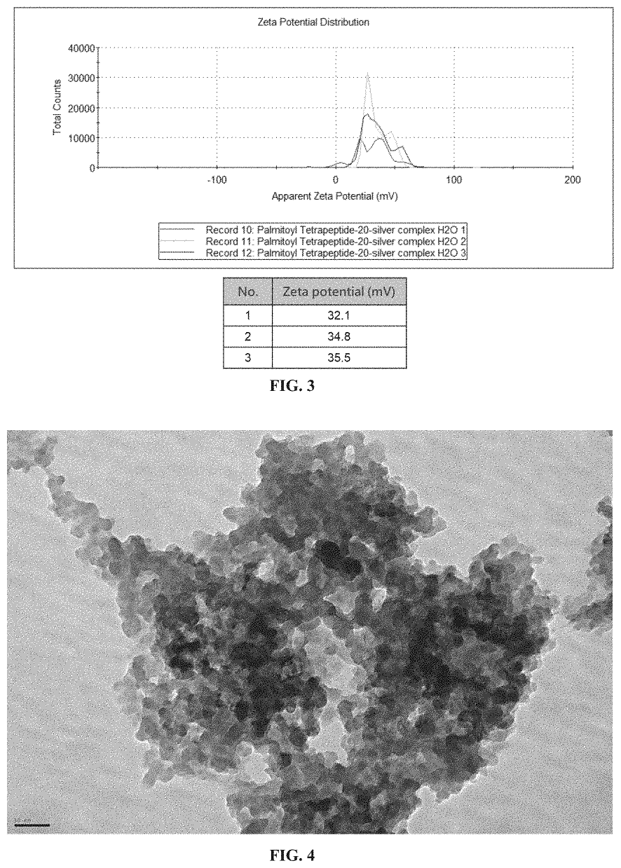

The data of particle size distribution is shown in FIG. 2, indicating that the palmitoyl tetrapeptide-20 conjugated nanosilver particle of the present invention is at nano size level, wherein the particle size may be within 10 nm to 200 nm, preferably within 10 nm to 150 nm, more preferably within 10 nm to 100 nm, more preferably within 10 nm to 90 nm, or even more preferably within 10 nm to 80 nm. Mean particle size of the three independent experiments is approximately 91.7±31.08 nm. The average of polydispersity index (PDI) is about 0.334±0.152.

After the particle is in equilibrium, it is found that the greater the electric potential difference between dispersant and dispersed particles, the easier it is to maintain a stable dispersion state. When the absolute value of electric potential difference is greater than 30 mV, it is in a stable dispersion state; when the absolute value of electric potential difference is less than 30 mV, the system is unstable and susceptible to aggregate; when the electric potential difference is 0, it represents isoelectric point. As shown in FIG. 3, the Zeta potential of the palmitoyl tetrapeptide-20 conjugated nanosilver particle of the present invention may be greater than +15 mV, preferably greater than +20 mV, more preferably greater than +30 mV, even more preferably between +30 mV and +38 mV. The mean of Zeta potential according to three independent experiments is approximately 34.1±1.8 mV, indicating that the palmitoyl tetrapeptide-20 conjugated nanosilver particle of the present invention has excellent stability and does not easily aggregate with each other and precipitate.

Observation of Transmission Electron Microscope (TEM) View

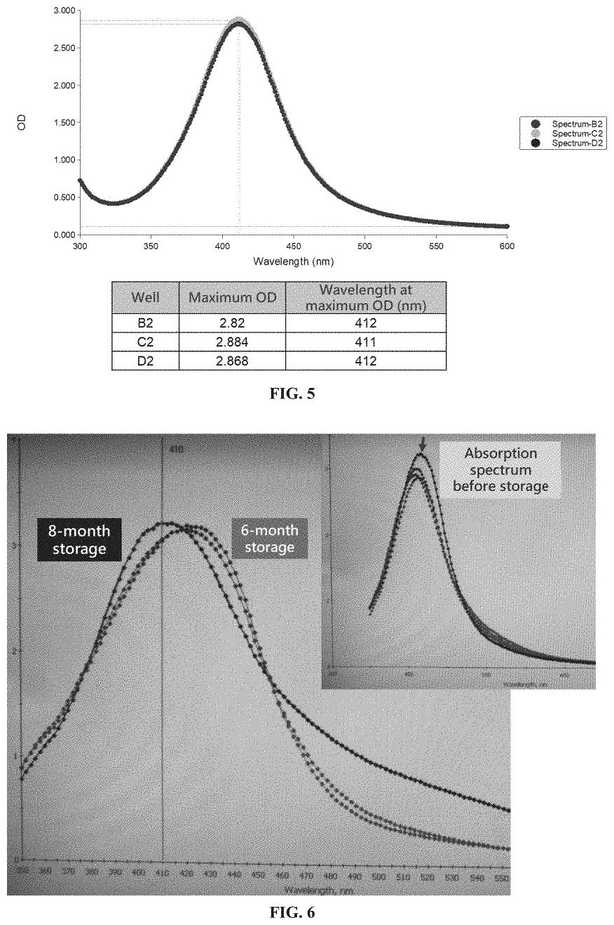

As shown in FIG. 4, the shape and size of palmitoyl tetrapeptide-20 conjugated nanosilver particle of the present invention is observed by a transmission electron microscope, wherein further confirms that its particle size is less than 100 nm and its appearance is a spherical nanoparticle.

Determination of Wavelength and Absorption Spectrum

The wavelength and absorption spectrum of the palmitoyl tetrapeptide-20 conjugated nanosilver particle in this invention is analyzed by an ultraviolet-visible spectrophotometer to determine whether the wavelength corresponding to the maximum absorption of the palmitoyl tetrapeptide-20 conjugated nanosilver particle is within the normal range of 395 nm to 415 nm. Take 0.1 mL of palmitoyl tetrapeptide-20 conjugated nanosilver particle, put it into a 96-well plate, and scan the absorption spectrum at a wavelength within 300 nm to 600 nm with Epoch™ 2 Microplate Spectrophotometer. The analysis is repeated for three independent experiments.

As shown in FIG. 5, the absorption wavelength of the palmitoyl tetrapeptide-20 conjugated nanosilver particle is preferably within 390 nm to 430 nm, more preferably within 395 nm to 425 nm, more preferably within 400 nm to 420 nm, even more preferably within 405 nm to 415 nm. There is a maximum absorption peak at a wavelength of about 412 nm.

Long-Term Assessment of Stability

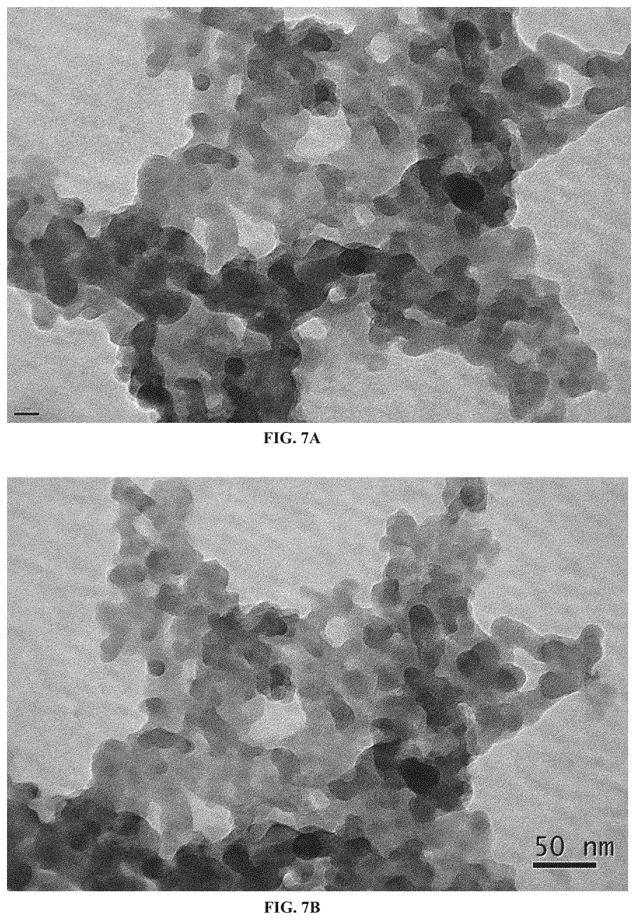

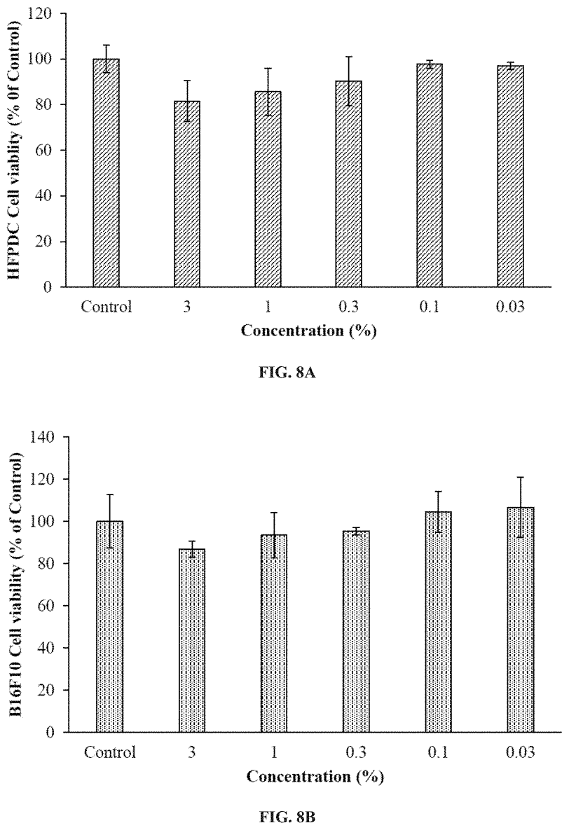

Two batches of palmitoyl tetrapeptide-20 conjugated nanosilver particle are produced at different time points. After being stored for 6 months and 8 months respectively, their respective absorption spectrum is analyzed by an ultraviolet-visible spectrophotometer. As shown in FIG. 6, with comparison to the absorption spectrum obtained prior to storage, the absorption spectrum of the palmitoyl tetrapeptide-20 conjugated nanosilver particle does not alter after 6-month and 8-month storage. The particle still presents a maximum absorption peak at a wavelength between 400 nm and 425 nm with an absorbance about 3.2, which still maintains high content. As shown in FIG. 7A and FIG. 7B, after long-term storage, the appearance of the palmitoyl tetrapeptide-20 conjugated nanosilver particle is observed by TEM, showing that after 6-month (FIG. 7A) and 8-month (FIG. 7B) storage, said particle is stable and maintains in a nanoscale morphology. Accordingly, the palmitoyl tetrapeptide-20 conjugated nanosilver particle of the present invention is stable for a long-term period, and does not deteriorate easily.

### EXAMPLES

The following examples are provided to better illustrate the nanocomposite material of the present invention, and the advantageous effects thereof through using the nanocomposite material of the present invention. It should be noted that these examples are not to be interpreted as limiting the scope of the claimed invention.

The concentrations of the palmitoyl tetrapeptide-20 conjugated nanosilver particle used in the following cellular assay are 0.03%, 0.1%, 0.3%, 1% and 3%, wherein the peptide concentration ranges from 10⁻⁸ M to 10⁻⁶ M.

Cell Culture

Human Hair Follicle Dermal Papilla Cells (HFDPC)

HFDPC are cultured in a complete cell culture medium in a 10-cm dish, which is pre-coated with 3 mL collagen coating solution, and then maintain the HFDPC cells in a cell culture incubator at 37° C. with 5% CO₂ atmosphere.

When the cells have reach 80% to 90% confluency, remove the used culture medium from the dish and wash the cells with HBSS. Add 5 mL of Trypsin-EDTA solution to cover the dish evenly, immediately remove 4.5 mL of Trypsin-EDTA solution from the dish, and incubated at room temperature for 3 minutes. Subsequently, add 5 mL trypsin neutralizing solution, and the cells will then detach from the dish. Collect the cells to a centrifuge tube for centrifugation. Then, remove the supernatant, add appropriate amount of culture medium, mix evenly, and seed the cells to a new culture dish for maintaining.

Murine Melanoma Cells (B16F10)

B16F10 cells are cultured in DMEM medium (containing 10% fetal bovine serum, 2 mM L-glutamine, 1 mM sodium pyruvate, 100 units/mL penicillin-G, and 100 μg/mL streptomycin sulfate) and maintained in a cell culture incubator at 37° C. with 5% CO₂ atmosphere.

The cells are passaged at 80% to 90% confluency. Remove used cell medium in the culture dish and wash the cells with PBS twice. The cells are treated with Trypsin-EDTA solution at 37° C. for 3 minutes, and then the cells will detach from the culture dish. Collect the cells to a centrifuge tube for centrifugation. Then, remove the supernatant, add appropriate amount of culture medium, mix evenly, and seed the cells to a new culture dish for maintaining.

### Example 1

### Cytotoxicity Assay

HFDPC Cell Line

HFDPC are seeded in 96-well plates at the density of 2×10⁴ cells/well with 100 μL of cell culture medium per well and incubated for 24 hours for cell attachment. After removing the used medium, the cells are treated with 200 μL of fresh culture medium (control group) or culture medium containing various concentrations of palmitoyl tetrapeptide-20 conjugated nanosilver particle, and incubated for 24 hours. At the end of treatment, cells are incubated with 5 mg/mL MTT solution at 37° C. for 1.5 hours. Subsequently, the solution is removed and replaced with 100 μL of DMSO to dissolve the purple formazan crystals at the bottom of the wells. Absorbance is determined at 570 nm (A_{570 nm}) using a microplate reader to calculate cell viability. The equation for calculating cell viability is described as following: Cell viability (%)=(A_{570 nm} treatment group/A_{570 nm} control group)×100%.

B16F10 Cell Line

B16F10 are seeded in 96-well plates at the density of 3×10⁴ cells/well and treated with various concentrations of palmitoyl tetrapeptide-20 conjugated nanosilver particle for 24 hours. At the end of treatment, cells are incubated with 5 mg/mL MTT solution at 37° C. for 40 minutes. Subsequently, the solution is removed and replaced with 100 μL of DMSO to fully dissolve the crystals at the bottom of the wells. Absorbance is determined at 570 nm using a microplate reader. Cell viability is calculated through the equation described above.

The experimental results are shown in FIGS. 8A and 8B. In FIG. 8A, the effect of various concentrations of palmitoyl tetrapeptide-20 conjugated nanosilver particle on the cell viability of HFDPC is shown. At low concentrations (0.03%, 0.1%), the cell viability of HFDPC is higher than 95%. The cell viability is about 90% with the treated concentration increased to 0.3%. With the treated concentration increased to 1% and 3%, the cell viability is 86% and 81% respectively. In FIG. 8B, the effect of various concentrations of palmitoyl tetrapeptide-20 conjugated nanosilver particle on the cell viability of B16F10 is shown. The cell viability of B16F10 is higher than 95% at a treated concentration from 0.03% to 0.3%, and is about 86% when the treated concentration increased to 3%. The results demonstrate that the palmitoyl tetrapeptide-20 conjugated nanosilver particle of the present invention is not cytotoxic and is a safe and effective hair care product.

### Example 2

### Measurement of Melanin Production

B16F10 are seeded in 24-well plates at the density of 8×10⁴ cells/well and incubated for 24 hours at 37° C. with 5% CO₂ atmosphere. After removing the used cell medium, the cells are treated with fresh cell culture medium (control group), 200 nM α-MSH (α-MSH group), combination of 200 nM α-MSH and various concentrations of palmitoyl tetrapeptide-20 conjugated nanosilver particle (α-MSH+sample group), and various concentrations of palmitoyl tetrapeptide-20 conjugated nanosilver particle (sample group), for 24 hours. After treatment, the treated solution in the well is removed with further incubation with fresh cell culture medium for 24 hours.

The cells are washed twice with PBS. The cells are transferred into a 1.5 mL microtube and centrifuged at 10,000 rpm for 5 minutes to obtain cell pellets. Add 100 μL of solution (1 N NaOH containing 10% DMSO) to the pellet for 60 minutes at 80° C. to dissolve intracellular melanin. Extract 80 μL of dissolved melanin and place in 96-well plates, and melanin contents of control group and treatment group are determined by measuring the absorbance at 405 nm. The assay is repeated for three independent experiments. The equation for calculating melanin content is described as following: Total melanin content (%)=(A_{405 nm} treatment group/A_{405 nm} control group)×100%.

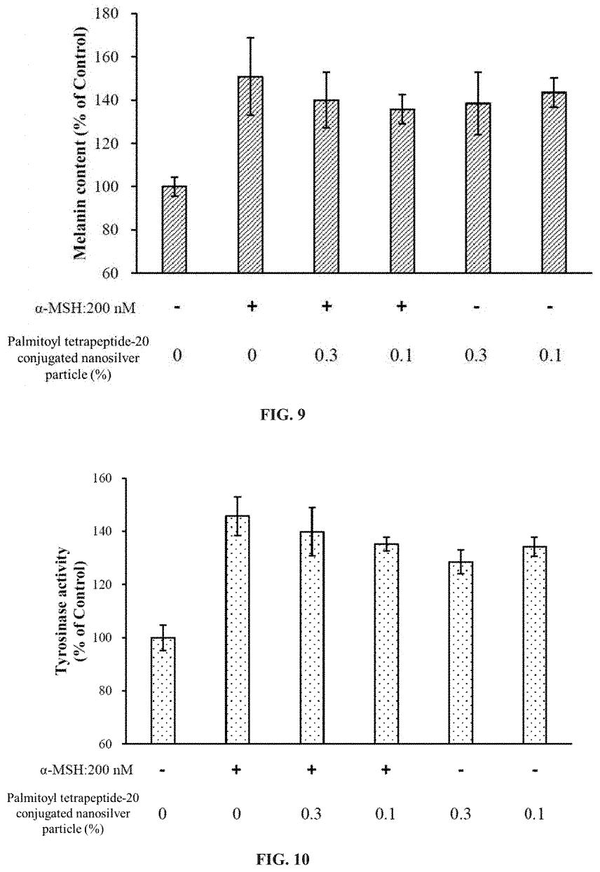

This assay compares the effects of co-treatment with α-MSH and palmitoyl tetrapeptide-20 conjugated nanosilver particle, and single treatment with α-MSH or palmitoyl tetrapeptide-20 conjugated nanosilver particle, on the melanogenesis in B16F10 cells. As shown in FIG. 9, α-MSH group, α-MSH+sample group, and sample group all effectively induce melanin synthesis, wherein no significant difference in the melanin content is found between these three groups. Single treatment with 0.1% or 0.3% of palmitoyl tetrapeptide-20 conjugated nanosilver particle (sample group) significantly increases melanin content by approximate 44% or 38% compared to control group. This result indicates that the palmitoyl tetrapeptide-20 conjugated nanosilver particle of the present invention can significantly promote melanogenesis in melanin-producing cells.

### Example 3

### Cellular Tyrosinase Activity Assay

Tyrosinase is a critical enzyme in melanogenic pathway, which catalyzes the first two steps of melanin synthesis, respectively: (1) catalyzing the hydroxylation of L-tyrosine to form L-DOPA; (2) subsequently catalyzing the oxidation of L-DOPA to form L-DOPAquinone. L-DOPAquinone is then converted to DOPAchrome through auto-oxidation, and then tyrosinase related proteins (TRP-1, TRP-2) catalyze a series of following reactions to form melanin.

Intracellular tyrosinase activity in B16F10 cells is examined by the following assay. B16F10 are seeded in 24-well plates at the density of 8×10⁴ cells/well and incubated for 24 hours at 37° C. with 5% CO₂ atmosphere. After removing the used cell medium, the cells are treated with fresh cell culture medium (control group), 200 nM α-MSH (α-MSH group), combination of 200 nM α-MSH and various concentrations of palmitoyl tetrapeptide-20 conjugated nanosilver particle (α-MSH+sample group), or various concentrations of palmitoyl tetrapeptide-20 conjugated nanosilver particle (sample group), for 24 hours. After treatment, the treated solution in the well is removed with additional incubation with fresh cell culture medium for 24 hours.

The cells are washed twice with cold PBS. The cells are transferred into a 1.5 mL microtube and centrifuged at 10,000 rpm for 5 minutes. The cell pellets are then lysed with 100 μL of PBS containing 1% Triton X-100 (pH 6.8) at −80° C. for 30 minutes, followed by thawing at room temperature for 30 minutes. After repeating freeze-thaw cycles 3 times, the lysate is centrifuged at 12,000 rpm for 10 minutes. 90 μL of supernatant and 20 μL of reaction substrate of 10 mM L-DOPA are added in the well of a 96-well plate. After the wells are incubated at 37° C. for 30 minutes in the dark, absorbance is measured at 475 nm by a microplate reader to determine the production of dopachrome. The experimental data is expressed as a percentage compared to the control group. This assay is repeated for three independent experiments. The equation for calculating cellular tyrosinase activity is described as following: Intracellular tyrosinase activity (%)=(A_{475 nm} treatment group/A_{475 nm} control group)×100%.

As the results shown in FIG. 10, the pattern is the same as that in the assay for intracellular melanin content in the above-mentioned Example 3. Single treatment with 0.1% or 0.3% of palmitoyl tetrapeptide-20 conjugated nanosilver particle (sample group) significantly promotes intracellular tyrosinase activity by approximate 34% or 29%. This result demonstrates that the palmitoyl tetrapeptide-20 conjugated nanosilver particle of the present invention can promote tyrosinase activity in melanin-producing cells, thereby activating melanin synthesis pathway and thus increasing melanin production.

### Example 4

### Analysis of the Expression Level of Melanogenic Proteins

In addition to the involvement of tyrosinase, TRP-1 and TRP-2 mentioned above in melanogenesis, microphthalmia-associated transcription factor (MITF) also plays a key role in melanin synthesis, as well as the occurrence and transport of melanosomes. MITF regulates the expression of enzymes related to melanogenesis such as tyrosinase, TRP-1 and TRP-2.

Binding of α-melanocyte stimulating hormone (α-MSH) to melanocortin 1 receptor (MC1R) on the cell membrane activates adenylate cyclase to convert ATP into cAMP, leading to an increase of cAMP in a cell serving as second messenger to activate protein kinase A. Subsequently, protein kinase A translocates to the nucleus where it phosphorylates CREB, leading to increase the expression of MITF. MITF binds to the promoter of genes responsible for pigment synthesis (tyrosinase, TRP-1 and TRP-2), which upregulates the expression of such genes, ultimately promoting melanin synthesis.

Therefore, the expression level of such melanogenic proteins will be examined by the following assay. B16F10 are seeded in 6-cm dishes at the density of 2×10⁵ cells and treated with cell culture medium (control group), 200 nM α-MSH (α-MSH group), combination of 200 nM α-MSH and various concentrations of palmitoyl tetrapeptide-20 conjugated nanosilver particle (α-MSH+sample group), or various concentrations of palmitoyl tetrapeptide-20 conjugated nanosilver particle (sample group). After treatment for 24 hours, the proteins in the cells are extracted by RIPA lysis buffer and then quantified. The expression of melanogenic proteins in B16F10 is analyzed by western blotting using TRP-1 and TRP-2 specific antibodies with the GAPDH as internal control.

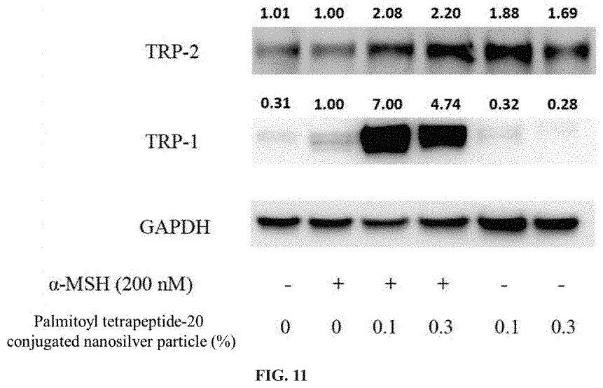

As shown in FIG. 11, after B16F10 are treated by the above four groups for 24 hours, the changes in the expression of melanogenic proteins (TRP-1 and TRP-2) are detected by western blot analysis. Protein expression levels of each group are expressed relative to α-MSH group, which is set as 1. The protein expression levels are digitally quantified using Image J software. Compared to single treatment with 200 nM α-MSH (α-MSH group), single treatment with 0.1% and 0.3% of palmitoyl tetrapeptide-20 conjugated nanosilver particle (sample group) respectively increases the protein expression level of TRP-2 to 1.88-fold and 1.69-fold of α-MSH group. In the co-treatment group of α-MSH and palmitoyl tetrapeptide-20 conjugated nanosilver particle (α-MSH+sample group), the cells are treated with 200 nM α-MSH in combination of 0.1% or 0.3% of palmitoyl tetrapeptide-20 conjugated nanosilver particle, resulting in further stimulation of protein expression level of TRP-2 to 2.08-fold and 2.20-fold of α-MSH group. Moreover, single treatment with palmitoyl tetrapeptide-20 conjugated nanosilver particle alone is unable to increase TRP-1 expression, but the protein expression level of TRP-1 is significantly increased to 7.00-fold and 4.74-fold of α-MSH group when the cells are co-treated with α-MSH.

This result shows that palmitoyl tetrapeptide-20 conjugated nanosilver particle of the present invention significantly improves the expression level of melanogenic proteins (TRP-1, TRP-2). In addition, it shows synergistic effects for α-melanocyte stimulating hormone when administrating palmitoyl tetrapeptide-20 conjugated nanosilver particle, which can further stimulate the melanogenesis.

### Example 5

### Hair Care Composition Improves the Synthesis of Natural Hair Pigments

Preparation of Hair Care Composition

Formulation of hair care composition is shown in Table 1. Each ingredient in Phase A is evenly mixed with each other followed by stirring for 5 to 10 minutes to complete hydration. Subsequently, ingredients in Phase B are added in order and mixed evenly. pH value is adjusted to 5.5 with citric acid. The product is then stored in a bottle and placed in dark at low temperature.

TABLE 1
Formulation of hair care composition

| Phase | Ingredient | Weight Percentage (%) |
|---|---|---|
| A | Water | 60.80 |
| | Glycerol | 5.00 |
| | D-Panthenol (Vitamin B5) | 0.50 |
| | Trimethylglycine | 1.00 |
| | Allantoin | 0.10 |
| B | Ethanol | 30.40 |
| | Peppermint Oil (Organic) | 0.20 |
| | Palmitoyl tetrapeptide-20 conjugated | 2.00 |
| | nanosilver particle | |

Efficacy Evaluation

The subjects, 20 in total, aged 20 to 65 containing males and females with hair graying issues are recruited. No hair dyeing is allowed within three weeks prior to testing. The hair care composition containing palmitoyl tetrapeptide-20 conjugated nanosilver particle is applied to the subjects every day after washing hair. The hair care composition is sprayed evenly on top of the scalp (8 times in total, approximately 2 mL) and massage scalp for 3 minutes to absorb. There is no need to wash afterwards. The treatment is 84 days in total, and the differences on the melanin synthesis in hair and hair lightness between before and after using hair care composition are evaluated. Measurement is performed before and after the treatment, the subjects stayed in a room with constant humidity of 50±10% and constant temperature at 20±2° C. for 15 minutes. Then, a Color reader (Konica Minolta's, CR-10) is used to determine the hair on the following locations: top, front hairline, sideburns at right and left sides. Meanwhile, conditions of the subjects' scalps and hairs are filmed and recorded with camera. After the treatment, ANOVA test and paired student's T test are performed to analyze the experiment results. When p-value<0.05, it indicates there is significant difference before and after the treatment.

Calibration Curve of Different Hair Color Levels Over Lightness L Values

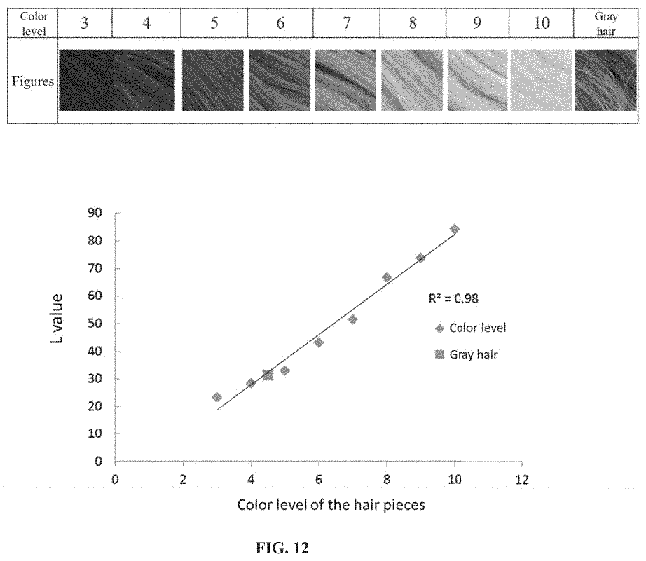

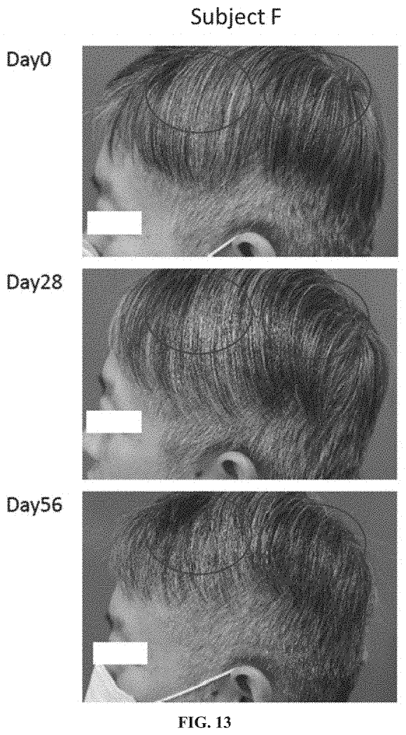

As shown in FIG. 12, a calibration curve of hair pieces with different hair color level over lightness L value is performed by linear regression analysis, with R²=0.98 indicating that lightness L value and hair color level are in highly linear relation. In general, color level of the gray hair of elderly is between 4 and 5.

Changes in Hair Color at Different Locations of Head After Treatment of Hair Care Composition

Left Side of the Head

As shown in FIG. 13 and Table. 2, after treatment with the hair care composition of the present invention for 28 days, darkening of hair color can be observed in 85% of the subjects, and the data also appears statistical significance compared to the hair color prior to treatment. After treatment for 84 days, darkening of hair color can be observed in 95% of the subjects with average hair darkening rate reaching 13.70%, and the data also shows statistical significance compared to the hair color before treatment, wherein 30% of the subjects (6 out of 20, i.e. 6/20) with hair darkening rate ranging between 10% and 20%, 35% of the subjects (7/20) with hair darkening rate over 20%.

TABLE 2
Changes in hair color at left side of the head after using the hair care composition

| | Darkening in | Hair darkening rate | Hair darkening rate | Average hair | |
|---|---|---|---|---|---|
| | hair color | between 10% and 20% | over 20% | darkening | |
| | (% of subjects) | (% of subjects) | (% of subjects) | rate | Significance |
| Day 28 | 85% | 20% | 0% | 5.70% | *** |
| | (17/20) | (4/20) | (0/20) | | |
| Day 56 | 100% | 50% | 5% | 10.60% | *** |
| | (20/20) | (10/20) | (1/20) | | |
| Day 84 | 95% | 30% | 35% | 13.70% | *** |
| | (19/20) | (6/20) | (7/20) | | |

Right Side of the Head

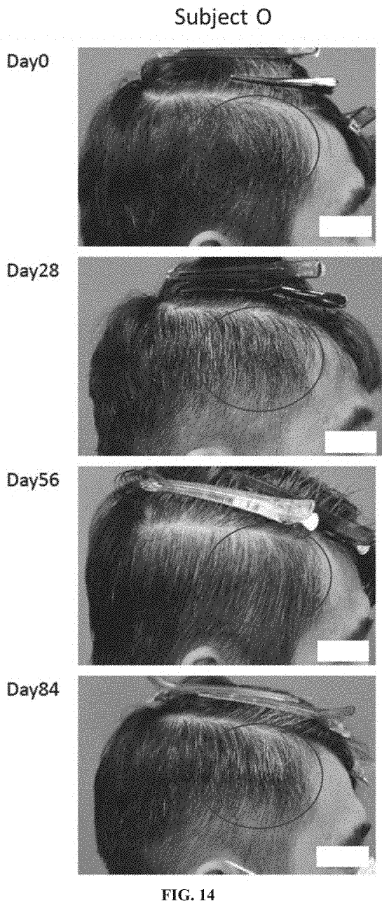

As shown in FIG. 14 and Table. 3, after treatment with the hair care composition of the present invention for 28 days, darkening of hair color can be observed in 90% of the subjects, and the data also appears statistical significance compared to the hair color prior to treatment. After treatment for 84 days, darkening of hair color can be observed in 100% of the subjects with average hair darkening rate reaching 13.70%, and the data also shows statistical significance compared to the hair color before treatment, wherein 45% of the subjects (9/20) with hair darkening rate ranging between 10% and 20%, 25% of the subjects (5/20) with hair darkening rate over 20%.

TABLE 3
Changes in hair color at right side of the head after using the hair care composition

| | Darkening in | Hair darkening rate | Hair darkening rate | Average hair | |
|---|---|---|---|---|---|
| | hair color | between 10% and 20% | over 20% | darkening | |
| | (% of subjects) | (% of subjects) | (% of subjects) | rate | Significance |
| Day 28 | 90% | 15% | 5% | 6.60% | *** |
| | (18/20) | (3/20) | (1/20) | | |
| Day 56 | 100% | 35% | 15% | 11.50% | *** |
| | (20/20) | (7/20) | (3/20) | | |
| Day 84 | 100% | 45% | 25% | 13.70% | *** |
| | (20/20) | (9/20) | (5/20) | | |

Top of the Head

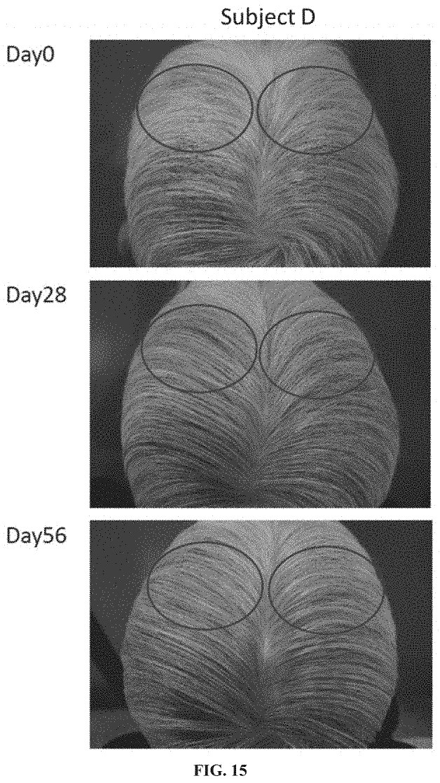

As shown in FIG. 15 and Table. 4, after treatment with the hair care composition of the present invention for 28 days, darkening of hair color can be observed in 85% of the subjects, and the data also appears statistical significance compared to the hair color prior to treatment. After treatment for 84 days, darkening of hair color can be observed in 90% of the subjects with average hair darkening rate reaching 10.90%, and the data also shows statistical significance compared to the hair color before treatment, wherein 40% of the subjects (8/20) with hair darkening rate ranging between 10% and 20%, 15% of the subjects (3/20) with hair darkening rate over 20%.

TABLE 4
Changes in hair color on top of the head after using the hair care composition

| | Darkening in | Hair darkening rate | Hair darkening rate | Average hair | |
|---|---|---|---|---|---|
| | hair color | between 10% and 20% | over 20% | darkening | |
| | (% of subjects) | (% of subjects) | (% of subjects) | rate | Significance |
| Day 28 | 85% | 15% | 0% | 4.40% | ** |
| | (17/20) | (3/20) | (0/20) | | |
| Day 56 | 80% | 30% | 10% | 8.40% | *** |
| | (16/20) | (6/20) | (2/20) | | |
| Day 84 | 90% | 40% | 15% | 10.90% | *** |
| | (18/20) | (8/20) | (3/20) | | |

Hairline

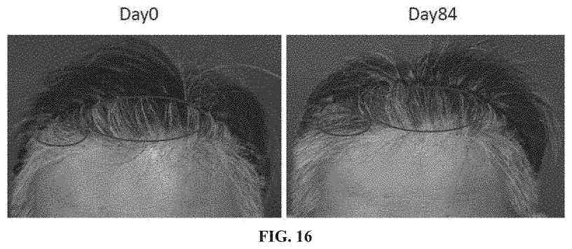

As shown in FIG. 16 and Table. 5, after treatment with the hair care composition of the present invention for 28 days, darkening of hair color can be observed in 80% of the subjects, and the data also appears statistical significance compared to the hair color prior to treatment. After treatment for 84 days, darkening of hair color can be observed in 80% of the subjects with average hair darkening rate reaching 8.80%, and the data also shows statistical significance compared to the hair color before treatment, wherein 20% of the subjects (4/20) with hair darkening rate ranging between 10% and 20%, 15% of the subjects (3/20) with hair darkening rate over 20%.

TABLE 5
Changes in hair color in hairline after using the hair care composition

| | Darkening in | Hair darkening rate | Hair darkening rate | Average hair | |
|---|---|---|---|---|---|
| | hair color | between 10% and 20% | over 20% | darkening | |
| | (% of subjects) | (% of subjects) | (% of subjects) | rate | Significance |
| Day 28 | 80% | 15% | 0% | 4.40% | ** |
| | (16/20) | (3/20) | (0/20) | | |
| Day 56 | 90% | 20% | 15% | 8.50% | *** |
| | (18/20) | (4/20) | (3/20) | | |
| Day 84 | 80% | 20% | 15% | 8.80% | *** |
| | (16/20) | (4/20) | (3/20) | | |

After using hair care composition of the present invention for 12 weeks by 20 subjects, the changes in hair color are shown in Table. 6. This result indicates that palmitoyl tetrapeptide-20 conjugated nanosilver particle of the present invention promotes melanin production in hair. The present invention darkens hair color and reverses the process of hair graying, thereby providing safe and effective hair care products.

TABLE 6
Changes in hair color after using the hair care composition for 12 weeks

| | Darkening in | Hair darkening rate | Hair darkening rate | Average hair | |
|---|---|---|---|---|---|
| | hair color | between 10% and 20% | over 20% | darkening | |
| Locations | (% of subjects) | (% of subjects) | (% of subjects) | rate | Significance |
| Left side | 95% | 30% | 35% | 13.70% | *** |
| | (19/20) | (6/20) | (7/20) | | |
| Right side | 100% | 45% | 25% | 13.70% | *** |
| | (20/20) | (9/20) | (5/20) | | |
| Top of the | 90% | 40% | 15% | 10.90% | *** |
| head | (18/20) | (8/20) | (3/20) | | |
| Hairline | 80% | 20% | 15% | 8.80% | *** |
| | (16/20) | (4/20) | (3/20) | | |

### Example 6

### Transdermal Delivery Analysis

Scalp absorption efficiency of palmitoyl tetrapeptide-20 conjugated nanosilver particle of the present invention is evaluated using Franz cells. The skin of pig ear is washed with ddH₂O, and the epithelium of the pig ear is obtained using a scalpel and then cut into a penetrating membrane with 1.5×1.5 cm² in area and 650 μm in thickness, followed by washing with PBS. The penetrating membrane is fixed to the Franz-type diffusion cell with diffusion area of 0.636 cm². 1.0 mL of tested sample (blank, palmitoyl tetrapeptide-20 conjugated nanosilver particle, palmitoyl tetrapeptide-20, nanosilver particle, unconjugated mixture of palmitoyl tetrapeptide-20 and nanosilver particle) is placed in donor compartment. 5.3 mL of PBS is filled in acceptor compartment. The experiment is conducted in a thermostatic bath at 32±1° C. with a stir bar to balance the concentration of the tested sample in donor compartment.

The result shows that palmitoyl tetrapeptide-20 cannot penetrate the skin, whereas high content of palmitoyl tetrapeptide-20 conjugated nanosilver particle penetrates more internal layers such as epidermis and dermis. Compared to the unconjugated mixture of palmitoyl tetrapeptide-20 and nanosilver particle, the palmitoyl tetrapeptide-20 conjugated nanosilver particle of the present invention increases cutaneous penetration efficiency by 20 to 80 times or more.

Therefore, the palmitoyl tetrapeptide-20 conjugated nanosilver particle of the present invention overcomes the poor skin absorption of palmitoyl tetrapeptide-20 by 20 to 80 times or more. The penetration of the nanocomposite material of the present invention can reach to the hair bulb, located at the bottom of hair follicle, and thus can more effectively stimulates the melanocytes to produce melanin, thereby improving the pigment concentration of new hair born from the hair follicle.

What is claimed:

1. A nanocomposite material, comprising a palmitoyl tetrapeptide-20 conjugated nanosilver particle formed by conjugation of palmitoyl tetrapeptide-20 and nanosilver particle.

2. The nanocomposite material of claim 1, wherein the palmitoyl tetrapeptide-20 conjugated nanosilver particle is analyzed in the form of nanosilver aqueous solution by an ultraviolet/visible spectrophotometer; an absorption spectrum has a specific absorption spectrum peak within 390 nm to 430 nm.

3. The nanocomposite material of claim 1, wherein a particle size of the palmitoyl tetrapeptide-20 conjugated nanosilver particle is within 10 nm to 200 nm.

4. The nanocomposite material of claim 1, wherein a Zeta potential of the palmitoyl tetrapeptide-20 conjugated nanosilver particle is greater than +15 mV.

5. The nanocomposite material of claims 1, wherein the palmitoyl tetrapeptide-20 conjugated nanosilver particle significantly increases the melanin content in newly born hair.

6. The nanocomposite material of claims 1, wherein the palmitoyl tetrapeptide-20 conjugated nanosilver particle increases cutaneous penetration.

7. A hair care composition, comprising the nanocomposite material of claim 1, and the hair care composition ameliorates the gray hair in a subject in need through allowing the newly born hair to recover to its original color.

8. A method of ameliorating gray hair in a subject in need, comprising the step of contacting the scalp of the subject with the nanocomposite material of claims 1, thereby increasing the melanin content in newly born hair.

9. A use of the nanocomposite material as defined in claim 1 for maintaining natural hair color or ameliorating gray hair appearance in a subject in need.
